(19) United States
(12) Patent Application Publication
(10) Pub. No.: US 2018/0165435 A1
(43) Pub. Date: Jun. 14, 2018

(54) CONTRACT CANCELLATION SYSTEM, SERVER, AND CONTRACT CANCELLATION METHOD

(71) Applicant: Panasonic Intellectual Property Management Co., Ltd., Osaka (JP)

(72) Inventors: NAOMI TOMIYAMA, Kyoto (JP); YUUMI KANEKO, Kanagawa (JP); YUICHI AOKI, Osaka (JP); MOTOJI OHMORI, Osaka (JP)

(21) Appl. No.: 15/823,574

(22) Filed: Nov. 28, 2017

(30) Foreign Application Priority Data
Dec. 13, 2016 (JP) 2016-241568

(51) Int. Cl. G06F 21/32; G06Q 50/18; G06Q 20/40; G06F 21/60; A61B 5/16

(52) U.S. Cl. CPC G06F 21/32; G06Q 50/188; A61B 5/165; G06F 21/602; G06Q 20/407

(57) ABSTRACT

A contract cancellation system includes a first communication terminal, a second communication terminal, and a server. The first communication terminal operated by a first user acquires biological data of the first user and transmits, to the server, contract history information associating contract information, time information, and the biological data. The second communication terminal operated by a second user transmits, to the server, a cancellation request notification for requesting cancellation of the contract procedure when the second user has operated to cancel the contract procedure. The server receives the contract history information, records the contract history information, and executes processing of the contract procedure. The server cancels the contract procedure when receiving the cancellation request notification within a first period and the biological data associated with the contract information deviates from an allowable range.

### BACKGROUND

### 1. Technical Field

The present disclosure relates to a contract cancellation system for canceling a prescribed contract procedure that has been performed by a user, a server for the contract cancellation system, and a contract cancellation method for the server. The present disclosure relates to a contract cancellation system for canceling an electronic settlement procedure or an electronic commerce procedure that has been performed by a user, a server for the contract cancellation system, and a contract cancellation method for the server, for example.

### 2. Description of the Related Art

In recent years, due to the growth of the Internet, it has been an everyday affair that a user uses a communication terminal owned by the user, an automated teller machine, or the like to perform an electronic settlement procedure such as remittance or transfer, or a user uses the communication terminal owned by the user to perform an electronic commerce procedure for purchasing a product or a service.

For example, with respect to the above-described electronic commerce procedure, Japanese Unexamined Patent Application Publication No. 2010-67066 discloses an information processing device that enables an operation through a network. This information processing device includes a measurement unit that detects a mental state of the user, a determination unit that determines whether measurement data detected by the measurement unit is within a preset normal value range, a control unit that allows an operation through the network when the measurement data is determined to be within the normal value range by the determination unit, and a warning unit that generates a prescribed warning when the measurement data is determined not to be within the normal value range by the determination unit. According to Japanese Unexamined Patent Application Publication No. 2010-67066, a mental state of a user can be grasped in order to let the user perform an operation in a normal state of mind.

Furthermore, Japanese Unexamined Patent Application Publication No. 11-328272 discloses an online shopping system that includes a subscription processing function with which a transaction using an automatic settlement terminal device is not actually settled for a certain number of days as a subscription period and is accepted as subscription of settlement and executes settlement processing after the number of days has elapsed. According to Japanese Unexamined Patent Application Publication No. 11-328272, the automatic settlement terminal device can be used to perform cancellation of product settlement.

Furthermore, Japanese Unexamined Patent Application Publication No. 10-320458 discloses using a mobile information terminal that includes a bidirectional wireless communication unit to provide a unit with which details of a transaction requested to a service company from a requestor using a communication tool such as the Internet are sent back to the requestor for the sake of confirmation using another communication tool from the service company, so that the requestor can confirm the details. According to Japanese Unexamined Patent Application Publication No. 10-320458, a safe electronic commerce system is implemented by providing a unit with which, in a commerce transaction using the Internet or the like, a party of the transaction holding the power of decision can confirm the details before a final settlement is made.

### SUMMARY

One non-limiting and exemplary embodiment provides a technique with which, when a user has performed a contract procedure by mistake in an inappropriate state of mind, the user can appropriately handle the contract procedure after performing the contract procedure.

In one general aspect, the techniques disclosed here feature a contract cancellation system including a first communication terminal; a second communication terminal; and a server. The first communication terminal includes a first operation part operated by a first user, an acquirer that acquires biological data of the first user, and a first transmitter that transmits, to the server, contract history information in which contract information indicating contract content of a contract procedure, time information indicating a time of performing the contract procedure, and the biological data at the time of performing the contract procedure are associated with one another, when the first user has performed the contract procedure on the first operation part. The second communication terminal includes a second operation part operated by a second user, and a second transmitter that transmits, to the server, a cancellation request notification for requesting cancellation of the contract procedure, when the second user has performed an operation for canceling the contract procedure on the second operation part. The server includes a first receiver that receives the contract history information, a recorder that records the contract history information received by the first receiver, and a contract processor that executes processing of the contract procedure after a first period has elapsed from a time of recording the contract history information by the recorder. The contract processor cancels the contract procedure, when the first receiver has received the cancellation request notification within the first period and the biological data associated with the contract information deviates from an allowable range.

It should be noted that a general or specific embodiment may be implemented as an element, a device, an apparatus, a system, an integrated circuit, a method, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a block diagram of an example of a configuration of a contract cancellation system according to an embodiment of the present disclosure;

FIG. 2 is a sequence diagram illustrating an example of contract cancellation processing executed by the contract cancellation system illustrated in FIG. 1; and

FIG. 3 is a flowchart illustrating an example of server contract cancellation processing executed by the server illustrated in FIG. 1.

### DETAILED DESCRIPTION

### (Underlying Knowledge Forming Basis of the Present Disclosure)

According to Japanese Unexamined Patent Application Publication No. 2010-67066, when blood pressure and pulse count of a user performing an operation through a network are not within a normal value range, a warning is issued to the user. However, there are some cases where the user does not follow the warning when the user is not in a normal state of mind. Japanese Unexamined Patent Application Publication No. 2010-67066 does not state anything about how to handle a case where the user who is not in a normal state of mind does not follow the warning as described above.

Furthermore, according to Japanese Unexamined Patent Application Publication No. 11-328272, a transaction using an automatic settlement terminal device is not actually settled for a certain number of days as a subscription period and is accepted as subscription of settlement. However, because settlement processing is executed after the subscription period has elapsed, even if the user simply argues after completion of the settlement processing that the contract procedure has been performed by mistake, it is not easy to invalidate the contract procedure.

Furthermore, in Japanese Unexamined Patent Application Publication No. 10-320458, details of a transaction requested to a service company are sent back to the requestor for the sake of confirmation using another communication tool from the service company. However, there are some cases where the user who is not in a normal state of mind agrees with the sent confirmation and the transaction is completed. When the user who is not in a normal state of mind has agreed with the confirmation to execute processing of the contract procedure, and after that the user simply argues that the contract procedure has been performed by mistake, it is not easy to invalidate the contract procedure.

On the other hand, in a case where a user simply argues that a contract procedure has been performed by mistake after executing the processing of the contract procedure, if a counterpart of the contract procedure such as a company unconditionally allows cancellation of all details of the contract procedure, the counterpart cannot smoothly execute the details of the contract for which the contract procedure has been performed. For this reason, in order to allow cancellation of the contract procedure after the contract procedure has been performed, requirements in an appropriate range for both of the user and the counterpart of the contract procedure as the parties of the contract need to be defined.

As described above, with the above-described conventional techniques, in a case where the user has performed a contract procedure by mistake in an inappropriate state of mind, nothing is considered about how to handle the contract procedure after the user has performed the contract procedure, and further improvements are thus needed about how to handle the contract procedure after the user has performed the contract procedure.

In recent years, measurement of brain waves, heart rates, blood pressures, gases discharged from body, and the like has become easy. Furthermore, it has become possible to non-invasively measure these pieces of biological data, and accurate estimation of a user's state of mind has become possible using these pieces of biological data. From this background, it is possible to detect that a user performing a prescribed contract procedure is in an excited state of mind and cannot make a normal determination, and to determine, in such a case, that the user has performed the contract procedure by mistake.

A mistake means an error or a fault and means that a fact does not coincide with an idea. It also means a matter that actually occurs does not coincide with an idea. Furthermore, a mistake means that a declarant himself or herself does not know that no internal effective intent corresponding to an external effective intent is present, and in civil law, when there is a mistake with respect to an element of a legal act, the act is invalidated.

Based on the above-described underlying knowledge, the present inventors has earnestly investigated requirements in an appropriate range for both of the user and the counterpart of a contract procedure as the parties of the contract, as requirements for allowing cancellation of the contract procedure after the contract procedure has been performed. The present inventors has also earnestly investigated a handling method thereof, which enable to appropriately handle the contract procedure after the user has performed the contract procedure. As a result, the present inventors have completed the present disclosure.

A contract cancellation system according to an aspect of the present disclosure includes a first communication terminal; a second communication terminal; and a server. The first communication terminal includes a first operation part operated by a first user, an acquirer that acquires biological data of the first user, and a first transmitter that transmits, to the server, contract history information in which contract information indicating contract content of a contract procedure, time information indicating a time of performing the contract procedure, and the biological data at the time of performing the contract procedure are associated with one another, when the first user has performed the contract procedure on the first operation part. The second communication terminal includes a second operation part operated by a second user, and a second transmitter that transmits, to the server, a cancellation request notification for requesting cancellation of the contract procedure, when the second user has performed an operation for canceling the contract procedure on the second operation part. The server includes a first receiver that receives the contract history information, a recorder that records the contract history information received by the first receiver, and a contract processor that executes processing of the contract procedure after a first period has elapsed from a time of recording the contract history information by the recorder. The contract processor cancels the contract procedure, when the first receiver has received the cancellation request notification within the first period and the biological data associated with the contract information deviates from an allowable range.

With this configuration, the first communication terminal acquires the biological data of the first user performing the contract procedure and transmits, to the server, the contract history information associating the contract information indicating contract content of the contract procedure input by the first user, the time information indicating the time of performing the contract procedure, and the biological data at the time of performing the contract procedure. The server receives the contract history information transmitted from the first communication terminal and records the received contract history information in the recorder. After a prescribed grace period has elapsed from the time of recording the contract history information in the recorder, processing of the contract procedure corresponding to the contract content indicated by the contract information included in the contract history information is executed.

When the second user performs the operation for canceling the contract procedure on the second communication terminal, a cancellation request notification requesting cancellation of the contract procedure input by the second user is transmitted to the server. When the server has received the cancellation request notification transmitted from the second communication terminal within the grace period, the server identifies the piece of contract information corresponding to the cancellation request notification from the contract history information recorded in the recorder. When the piece of biological data associated with the identified piece of contract information deviates from a prescribed allowable range, the contract procedure is canceled in accordance with the cancellation request notification.

Accordingly, only when the biological data of the user performing the contract procedure deviates from a prescribed allowable range, the contract procedure is canceled after the contract procedure has been performed. With this configuration, as requirements in an appropriate range for both of the user and the counterpart of the contract procedure, only when it is possible to certify that the user has performed the contract procedure by mistake in an inappropriate state of mind, the contract procedure can be canceled after the user has performed the contract procedure. Consequently, when the user has performed the contract procedure by mistake in an inappropriate state of mind, the contract procedure can be appropriately handled after the user has performed the contract procedure.

In the above-described contract cancellation system, the contract procedure may be an electronic settlement procedure, and the contract processor may cancel the electronic settlement procedure, when the first receiver has received the cancellation request notification within the first period and the biological data associated with the contract information deviates from the allowable range.

With this configuration, in an electronic settlement procedure such as remittance or transfer, only when the biological data of the user performing the electronic settlement procedure deviates from a prescribed allowable range, the electronic settlement procedure is canceled after the electronic settlement procedure has been performed. Accordingly, as requirements in an appropriate range for both of the user and a trader such as a bank that executes processing of the electronic settlement procedure, only when it is possible to certify that the user has performed the electronic settlement procedure by mistake in an inappropriate state of mind, the electronic settlement procedure can be canceled after the user has performed the electronic settlement procedure.

In the above-described contract cancellation system, the contract procedure may be an electronic commerce procedure, and the contract processor may cancel the electronic commerce procedure, when the first receiver has received the cancellation request notification within the first period and the biological data associated with the contract information deviates from the allowable range.

With this configuration, in an electronic commerce procedure such as buying and selling of a product or a service (for example, online shopping, online trading, auction service, and digital content trading), only when the biological data of the user performing the electronic commerce procedure deviates from a prescribed allowable range, the electronic commerce procedure is canceled after the electronic commerce procedure has been performed. Accordingly, as requirements in an appropriate range for both of the user and a trader that executes processing of the electronic commerce procedure, only when it is possible to certify that the user has performed the electronic commerce procedure by mistake in an inappropriate state of mind, the electronic commerce procedure can be canceled after the user has performed the electronic commerce procedure.

In the above-described contract cancellation system, the server may further include an estimator that estimates a state of excitement of the first user by analyzing the biological data, and the contract processor may cancel the contract procedure, when the first receiver has received the cancellation request notification within the first period and the estimator estimates that the first user has been in a state of excitement, based on the biological data associated with the contract information.

With this configuration, when the cancellation request notification is received within the grace period, the piece of contract information corresponding to the cancellation request notification is identified from the contract history information recorded in the recorder. When it is estimated, from the piece of biological data associated with the identified piece of contract information, that the first user has been in a state of excitement, the contract procedure is canceled in accordance with the cancellation request notification. Accordingly, only when it is estimated, from the biological data of the user performing the contract procedure, that the user has been in a state of excitement, the contract procedure is canceled after the contract procedure has been performed. With this, only when it is accurately detected that the user has performed the contract procedure by mistake in an inappropriate state of mind, the contract procedure can be canceled after the user has performed the contract procedure.

In the above-described contract cancellation system, the estimator may estimate that the first user has been in a state of excitement, when a difference between first reference biological data being an average value of biological data of the first user in a normal state and the biological data is equal to or higher than a prescribed value.

With this configuration, average biological data obtained by averaging pieces of biological data of the first user in a normal state is acquired, and when a difference between the average biological data and the piece of biological data recorded in the recorder is equal to or higher than a prescribed value, it is estimated that the first user has been in a state of excitement. This enables to more accurately detect that the first user has performed the contract procedure by mistake in an inappropriate state of mind.

In the above-described contract cancellation system, the estimator may estimate that the first user has been in a state of excitement, when a difference between second reference biological data being an average value of biological data of a plurality of users in a normal state and the biological data is equal to or higher than a prescribed value.

With this configuration, general average biological data obtained by averaging pieces of biological data of general users in a normal state is acquired, and when a difference between the general average biological data and the piece of biological data recorded in the recorder is equal to or higher than a prescribed value, it is estimated that the first user has been in a state of excitement. This enables to more accurately detect that the first user has performed the contract procedure by mistake in an inappropriate state of mind.

In the above-described contract cancellation system, the contract processor may create a cancellation confirmation notification for confirming whether to cancel the contract procedure with the second user after a second period has elapsed from the time of recording the contract history information by the recorder, when the biological data associated with the contract information deviates from the allowable range. The server further may include a third transmitter that transmits the cancellation confirmation notification to the second communication terminal. The second communication terminal further may include a second receiver that receives the cancellation confirmation notification. The second transmitter may transmit, to the server, a cancellation reply notification for requesting cancellation of the contract procedure, when the second user has performed an operation for canceling the contract procedure on the second operation part in response to the cancellation confirmation notification, the second transmitter transmits, to the server. The contract processor may cancel the contract procedure, when the first receiver has received the cancellation reply notification.

With this configuration, in a case where the piece of biological data of the first user performing the contract procedure that is recorded in the recorder deviates from the allowable range, when a prescribed waiting period has elapsed from the time of recording the piece of biological data deviating from the allowable range, the server creates the cancellation confirmation notification for confirming whether to cancel the corresponding contract procedure with the second user and transmits the cancellation confirmation notification to the second communication terminal. The second communication terminal receives the cancellation confirmation notification and, when the second user requests cancellation of the contract procedure in response to the cancellation confirmation notification, transmits, to the server, the cancellation reply notification for requesting cancellation of the contract procedure. When the server has received the cancellation reply notification, the contract procedure is canceled in response to the cancellation reply notification.

As described above, even when the cancellation request notification is not transmitted from the second communication terminal, the cancellation confirmation notification is automatically transmitted from the server to the second communication terminal. Accordingly, the cancellation reply notification in response to the cancellation confirmation notification is transmitted from the second communication terminal, whereby only when it is possible to certify that the first user has performed the contract procedure by mistake in an inappropriate state of mind, the contract procedure can be canceled after the first user has performed the contract procedure.

In the above-described contract cancellation system, the second transmitter may transmit, to the server, an execution request notification requesting execution of the contract procedure, when the second user has performed an operation for requesting execution of the contract procedure on the second operation part in response to the cancellation confirmation notification. The contract processor may execute the processing of the contract procedure, when the contract processor has not received the cancellation reply notification from the second communication terminal within the second period from the time of transmitting the cancellation confirmation notification to the second communication terminal or has received the execution request notification from the second communication terminal.

With this configuration, in a case where the cancellation reply notification has not been received from the second communication terminal within a prescribed reconfirmation period after the cancellation confirmation notification is transmitted to the second communication terminal or the execution request notification has been received from the second communication terminal, processing of the contract procedure is executed. Accordingly, when execution of the processing of the contract procedure is implicitly desired without transmitting the cancellation reply notification or execution of the processing of the contract procedure is explicitly desired, the processing of the contract procedure can be promptly executed in accordance with the desire.

In the above-described contract cancellation system, the contract processor may create a cancellation confirmation notification for confirming whether to cancel the contract procedure with the first user, when the biological data associated with the contract information deviates from the allowable range. The server further may include a third transmitter that transmits the cancellation confirmation notification to the first communication terminal. The first communication terminal further may include a third receiver that receives the cancellation confirmation notification. The first transmitter may transmit, to the server, a cancellation reply notification for requesting cancellation of the contract procedure, when the first user has performed an operation for canceling the contract procedure on the first operation part in response to the cancellation confirmation notification. The contract processor may cancel the contract procedure, when the first receiver has received the cancellation reply notification.

With this configuration, in a case where the biological data of the first user performing the contract procedure that is recorded in the recorder deviates from the allowable range, the server creates the cancellation confirmation notification for confirming whether to cancel the corresponding contract procedure with the first user and transmits the cancellation confirmation notification to the first communication terminal. The first communication terminal receives the cancellation confirmation notification and, when the first user requests cancellation of the contract procedure in response to the cancellation confirmation notification, transmits, to the server, the cancellation reply notification for requesting cancellation of the contract procedure. When the server has received the cancellation reply notification, the contract procedure is canceled in response to the cancellation reply notification.

As described above, when the biological data of the first user performing the contract procedure deviates from the allowable range, the cancellation confirmation notification is automatically transmitted to the first communication terminal from the server. This enables to confirm whether to cancel the contract procedure with the first user. In response to this confirmation, the cancellation reply notification is transmitted from the first communication terminal, whereby the contract procedure can be canceled.

In the above-described contract cancellation system, the first communication terminal and the second communication terminal may be the same communication terminal. With this configuration, by using the same communication terminal, the contract procedure and cancellation thereof can be performed.

In the above-described contract cancellation system, the first user and the second user may be the same user. With this configuration, a user being a party of the contract can perform the contract procedure and cancellation thereof based on the user's determination.

In the above-described contract cancellation system, the acquirer may acquire the biological data from a wearable terminal worn by the first user.

With this configuration, the biological data can be acquired from the wearable terminal worn by the first user, enabling to acquire accurate biological data of the first user.

In the above-described contract cancellation system, the acquirer may include a sensor for measuring the biological data.

With this configuration, the biological data can be measured from the sensor, enabling to acquire accurate biological data of the first user without using any other device such as a wearable terminal.

A server according to another aspect of the present disclosure is communicably connected to a first communication terminal and a second communication terminal and includes a receiver that receives, from the first communication terminal, contract history information in which contract information indicating contract content of a contract procedure performed by a first user using the first communication terminal, time information indicating a time of performing the contract procedure, and biological data of the first user at the time of performing the contract procedure are associated with one another; a recorder that records the contract history information received by the receiver, and a contract processor that executes processing of the contract procedure after a first period has elapsed from a time of recording the contract history information by the recorder. The contract processor cancels the contract procedure, when the receiver has received the cancellation request notification for requesting cancellation of the contract procedure within the first period from the second communication terminal and the biological data associated with the contract information deviates from an allowable range.

Furthermore, the present disclosure can be implemented not only as a contract cancellation system or a server that includes a characteristic configuration as described above but also as a contract cancellation method for executing characteristic processing corresponding to the characteristic configuration included in the contract cancellation system or the server, for example. Accordingly, another aspect described below also enables to attain the same effect as in the above-described contract cancellation system.

A contract cancellation method according to another aspect of the present disclosure includes: receiving contract history information in which contract information indicating contract content of a contract procedure performed by a first user, time information indicating a time of performing the contract procedure, and biological data of the first user at the time of performing the contract procedure are associated with one another, recording the received contract history information in a recorder, canceling the contract procedure, when a cancellation request notification by a second user for requesting cancellation of the contract procedure has been received within a first period from a time of recording the contract history information in the recorder and the biological data associated with the contract information deviates from an allowable range, and executing the contract procedure after a first period has elapsed, when the cancellation request notification has not been received within the first period.

Furthermore, the present disclosure may be implemented as a computer program causing a computer to execute characteristic processing included in the above-described contract cancellation method. Needless to say, such a computer program can be distributed via a non-transitory computer-readable recording medium such as a CD-ROM or a communication network such as the Internet.

It is to be noted that the embodiment described below indicates a specific example of the present disclosure. A numerical value, a shape, a component, a step, an order of steps, or the like indicated in the embodiment described below merely is an example and is not intended to limit the present disclosure. Furthermore, out of the components in the embodiment described below, any component that is not described in an independent claim indicating the uppermost concept is described as an optional component. Furthermore, details in all embodiments can be combined in any manner.

### Embodiment

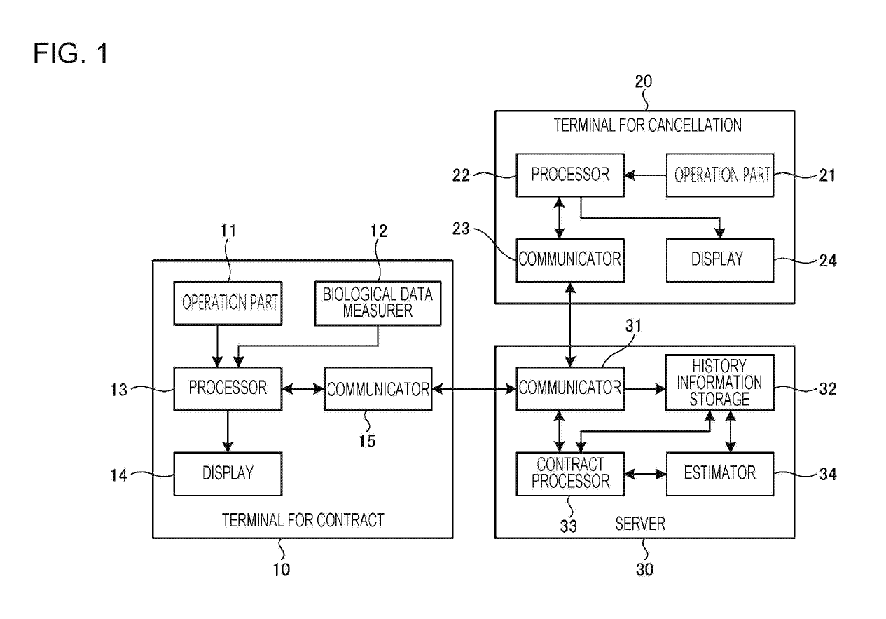

An embodiment according to the present disclosure will be described below with reference to the drawings. FIG. 1 is a block diagram of an example of a configuration of a contract cancellation system according to an embodiment of the present disclosure. The contract cancellation system illustrated in FIG. 1 includes a terminal 10 for contract, a terminal 20 for cancellation, and a server 30.

The terminal 10 for contract is a communication terminal (first communication terminal) used by a user (first user) being a party of a contract for performing a contract procedure. The terminal 20 for cancellation is a communication terminal (second communication terminal) used by the user being a party of the contract or a guardian (second user) such as a family member or a lawyer who manages the contract procedure of the user for canceling the contract procedure. The server 30 processes information related to the contract procedure performed by the user using the terminal 10. The terminal 10 and the terminal 20 are communicably connected to the server 30 via a prescribed wired or wireless network such as the Internet or a private line.

The contract procedure includes an electronic settlement procedure and an electronic commerce procedure, for example. The electronic settlement procedure includes processes such as remittance or transfer, and the electronic commerce procedure includes processes such as buying and selling of a product or a service, for example, processes of online trading, online shopping, auction service, digital content trading, and the like. Furthermore, a party of the contract procedure is a private person whose biological data can be acquired, and a counterpart of the contract procedure is a company such as a bank or a store, or a private person.

For example, in an electronic commerce procedure, there are some cases where the terminal 10 and the terminal 20 are the same communication terminal formed of a personal computer, for example, that is used by the user. In other cases, the terminal 10 is formed of a personal computer, for example, that is used by the user, and the terminal 20 is formed by a personal computer, for example, that is used by a guardian. These may be the same communication terminal or different communication terminals.

Furthermore, in an electronic settlement procedure, there are some cases where the terminal 10 is formed by an automated teller machine, for example, that is installed in a bank or the like, and the terminal 20 is formed of a stationary or mobile personal computer, for example, that is used by the user. These thus may be different communication terminals. In other cases, the terminal 10 and the terminal 20 are formed of a stationary or mobile personal computer, for example, that is used by the user. These thus may be the same communication terminal. In still other cases, the terminal 10 is formed of a stationary or mobile personal computer, for example, that is used by the user, and the terminal 20 is formed by a stationary or mobile personal computer, for example, that is used by a guardian. These may be the same communication terminal or different communication terminals.

It is to be noted that the terminal 10 and the terminal 20 are not especially limited to a communication terminal such as a personal computer as described above, and a mobile terminal such as a tablet or a smartphone may be used.

The terminal 10 includes an operation part 11, a biological data measurer 12, a processor 13, a display 14, and a communicator 15. The terminal 20 includes an operation part 21, a processor 22, a communicator 23, and a display 24. The server 30 includes a communicator 31, a history information storage 32, a contract processor 33, and an estimator 34.

The operation part 11 of the terminal 10 is formed of a keyboard, a mouse, or the like and operated by the user for performing the contract procedure. Specifically, the operation part 11 is used for inputting contract information indicating contract content of the contract procedure and outputs the contract information input by the user to the processor 13. It is to be noted that the configuration of the operation part 11 is not especially limited to the above-described example, and various modifications are possible. For example, an input device such as a touch panel may be used, or a microphone or the like may be used to input the contract information by voice recognition of the user's voices. Furthermore, the contract information may include identification information that identifies the user (for example, the user ID).

The biological data measurer 12 is formed of a sensor or the like that measures biological data, for example, measures biological data of the user performing the contract procedure by operating the operation part 11 to acquire the biological data, and outputs the acquired biological data to the processor 13. Brain waves, biological gases, blood pressures, respiration, heart rates, and/or body temperatures of the user correspond to the biological data, for example. The biological data measurer 12 includes a sensor for measuring pulse waves of a person in a non-contact manner using a high-sensitivity spectrum diffusion millimeter-wave radar or the like and detects heart rates and the like of the user, for example.

It is to be noted that the configuration of the biological data measurer 12 is not especially limited to this example. The user may wear a wearable terminal such as a smartwatch for measuring biological data of the user to acquire the biological data from the wearable terminal. In such a case, the biological data measurer 12 becomes a biological data acquirer for acquiring the biological data measured by the wearable terminal.

Furthermore, the biological data acquired by the biological data measurer 12 also is not especially limited to the above-described examples. As long as a state of excitement of the user can be estimated, which will be described later, other types of biological data such as voices, a face image, and a blood oxygen concentration of the user may be used. In such a case, the biological data measurer 12 is configured to measure those types of biological data.

The processor 13 generates contract history information associating contract information indicating the contract content of the contract procedure input by the user operating the operation part 11, time information indicating the time of performing the contract procedure that has been acquired by using a timer (not illustrated) inside the processor 13, and the biological data at the time of performing the contract procedure and outputs the generated contract history information to the communicator 15.

The communicator 15 performs communication between the server 30 and the terminal 10, transmits various types of information generated by the terminal 10 to the communicator 31, and receives various types of information generated by the server 30 from the communicator 31. Specifically, the communicator 15 transmits the contract history information to the server 30. It is to be noted that the configuration of the communicator 15 is not especially limited to the above-described example, and various modifications are possible. For example, the communicator 15 can be separated into a transmitter for transmitting information and a receiver for receiving information.

The display 14 is formed of a display device or the like, for example, and displays various operation screens and the like for the user to perform the contract procedure by operating the operation part 11.

The communicator 31 of the server 30 receives the contract history information transmitted from the terminal 10. The history information storage 32 records, as a recorder, the contract history information received by the communicator 31. It is to be noted that a method with which the history information storage 32 records the contract history information is not especially limited to the above-described example, and various modifications are possible. For example, the communicator 31 outputs the contract history information to the estimator 34, and the estimator 34 records, in the history information storage 32, only the piece of the contract history information that includes biological data with which the state of mind of the user at the time of performing the contract procedure is a state of excitement.

The contract processor 33 executes processing of contract procedure corresponding to the contract content indicated by the contract information included in the contract history information after a prescribed grace period has elapsed form the recording time when the contract history information is recorded in the history information storage 32. As the grace period, a company, such as a bank or a store, or a private person being the counterpart of the contract procedure may set an appropriate period, and a cooling-off period of 8 days, 10 days, 14 days, or 20 days from the day of the contract procedure, for example, may be used. Furthermore, as the grace period, a period until the processing of the contract procedure is actually executed may be used. Alternatively, even after the processing of the contract procedure is actually executed, when the executed processing can be canceled, a period after the processing of the contract procedure is actually executed may be used.

The operation part 21 of the terminal 20 is formed of a keyboard, a mouse, or the like and operated by the user (or the guardian) for performing cancellation of the contract procedure. Specifically, the operation part 21 is used for creating a cancellation request notification for requesting cancellation of the contract procedure and instructs the processor 22 to create the cancellation request notification. It is to be noted that the configuration of the operation part 21 is not especially limited to the above-described example, and various modifications are possible. For example, an input device such as a touch panel may be used, or a microphone or the like may be used to instruct creation of the cancellation request notification by voice recognition of the user's voices.

The processor 22 creates the cancellation request notification in accordance with an instruction from the operation part 21 and outputs the created cancellation request notification to the communicator 23. The cancellation request notification may include the contract information that identifies the contract procedure that the user desires to cancel, and may include identification information that identifies the user (for example, the user ID).

The communicator 23 performs communication between the server 30 and the terminal 20, transmits various types of information generated by the terminal 20 to the communicator 31, and receives various types of information generated by the server 30 from the communicator 31. Specifically, the communicator 23 transmits the cancellation request notification to the server 30. It is to be noted that the configuration of the communicator 23 is not especially limited to the above-described example, and various modifications are possible. For example, the communicator 23 can be separated into a transmitter for transmitting information and a receiver for receiving information.

The display 24 is formed of a display device or the like, for example, and displays various operation screens and the like for the user to transmit the cancellation request notification by operating the operation part 21.

The communicator 31 of the server 30 receives the cancellation request notification transmitted from the terminal 20 and outputs the received cancellation request notification to the contract processor 33. It is to be noted that the configuration of the communicator 31 is not especially limited to the above-described example, and various modifications are possible. For example, the communicator 31 can be separated into a transmitter for transmitting information and a receiver for receiving information.

When the communicator 31 has received the cancellation request notification within the grace period, the contract processor 33 identifies the piece of contract information corresponding to the contract procedure whose cancellation is requested by the cancellation request notification from the contract history information recorded in the history information storage 32. When the piece of biological data associated with the identified piece of contract information deviates from a prescribed allowable range, the contract processor 33 cancels the contract procedure in accordance with the cancellation request notification. It is to be noted that damage due to cancellation of the contract procedure may be borne by a company, such as a bank or a store, or a private person being the counterpart of the contract procedure, or a part of the damage may be bore by the user being a party of the contract procedure. Alternatively, an insurance system with respect to damage due to cancellation of the contract procedure may be established, so that the damage due to cancellation of the contract procedure is covered by an insurance premium.

Specifically, when the communicator 31 has received the cancellation request notification within the grace period, the contract processor 33 identifies the piece of contract information corresponding to the contract procedure whose cancellation is requested by the cancellation request notification from the contract history information recorded in the history information storage 32. The contract processor 33 then notifies the estimator 34 of the identified piece of contract information and makes an inquiry to the estimator 34 about the state of mind of the user at the time of performing the contract procedure.

The estimator 34 analyzes the biological data recorded in the history information storage 32, thereby estimating whether the state of mind of the user at the time of performing the contract procedure is a state of excitement from the piece of biological data associated with the identified piece of contract information, and notifies the contract processor 33 of the estimation result.

Specifically, the estimator 34 calculates a degree of excitement from the biological data recorded in the history information storage 32, for example, pulse waves of the user (for example, see Japanese Unexamined Patent Application Publication No. 11-39564) and, when the degree of excitement is equal to or higher than a prescribed value, determines that the user is in a state of excitement and estimates the state of excitement of the user. When the estimator 34 has estimated a state of excitement of the user, the contract processor 33 cancels the contract procedure in accordance with the cancellation request notification. It is to be noted that the state of mind of the user estimated by the estimator 34 is not especially limited to the above-described case, and may be other state of mind such as a panicked state or a depressed state as long as that state of mind is an inappropriate state in which the user can be certified as having performed the contract procedure by mistake.

Furthermore, when the server 30 can collect pieces of biological data of the user in a daily and normal state from a wearable terminal or the like worn by the user, the contract processor 33 may acquire the pieces of biological data of the user in a normal state using the communicator 31 and average the acquired pieces of biological data to calculate averaged biological data. In such a case, the estimator 34 may acquire the averaged biological data obtained by averaging the pieces of biological data of the user in a normal state from the contract processor 33 and, when a difference between the averaged biological data and the biological data recorded in the history information storage 32 is equal to or higher than a prescribed value, estimate that the user is in a state of excitement.

Furthermore, when the server 30 can collect pieces of biological data of a plurality of general users in a daily and normal state from wearable terminals or the like worn by the plurality of general users, the contract processor 33 may acquire the pieces of biological data of the plurality of general users in a normal state and average the acquired pieces of biological data to calculate averaged general biological data. In such a case, the estimator 34 may acquire the averaged general biological data obtained by averaging the pieces of biological data of the general users in a normal state from the contract processor 33 and, when a difference between the averaged general biological data and the biological data recorded in the history information storage 32 is equal to or higher than a prescribed value, estimate that the user is in a state of excitement.

It is to be noted that a method with which a state of excitement of the user is estimated using biological data is not especially limited to the above-described example, and various modifications are possible. For example, a state of excitement may be estimated from respiration of the user (see Japanese Unexamined Patent Application Publication No. 2008-289789) or estimated from blood flows of the user (see Japanese Unexamined Patent Application Publication No. 2005-46305 or Japanese Unexamined Patent Application Publication No. 2016-7414).

Furthermore, the server 30 may transmit a cancellation confirmation notification to the terminal 20. In such a case, the contract processor 33 makes an inquiry to the estimator 34 about whether the biological data of the user performing the contract procedure that is recorded in the history information storage 32 deviates from an allowable range. When the piece of biological data of the user performing the contract procedure that is recorded in the history information storage 32 deviates from an allowable range, the contract processor 33 creates the cancellation confirmation notification for confirming, with the user, whether to cancel the contract procedure indicated by the piece of contract information recorded in association with the deviating piece of biological data when a prescribed waiting period has elapsed from the deviating biological data recording time when the deviating piece of biological data deviating from the allowable range is recorded. The communicator 31 transmits the created cancellation confirmation notification to the terminal 20.

The cancellation confirmation notification may include the contract information that identifies the contract procedure to be canceled, and may include identification information that identifies the user (for example, the user ID). Furthermore, the cancellation confirmation notification is a notification for confirming cancellation of the contract procedure with the user before the grace period has elapsed, and an optional period thus may be set to the waiting period as long as the waiting period is shorter than the grace period.

The communicator 23 of the terminal 20 receives the cancellation confirmation notification from the server 30 and outputs the received cancellation confirmation notification to the processor 22. The processor 22 displays the cancellation confirmation notification on the display 24 to present the cancellation confirmation notification to the user (or the guardian).

When requesting cancellation of the contract procedure indicated by the piece of contract information recorded in association with the deviating piece of biological data in response to the cancellation confirmation notification, the user (or the guardian) operates the operation part 21 to make a reply with a cancellation reply notification in response to the cancellation confirmation notification. At this time, the processor 22 receives an instruction to make a reply with the cancellation reply notification from the operation part 21, and creates the cancellation reply notification for requesting cancellation of the contract procedure. The communicator 23 transmits the created cancellation reply notification to the server 30. The cancellation confirmation notification may include the contract information that identifies the contract procedure to be canceled, and may include identification information that identifies the user (for example, the user ID).

The communicator 31 of the server 30 receives the cancellation confirmation notification and outputs the received cancellation confirmation notification to the contract processor 33. When the communicator 31 has received the cancellation reply notification, the contract processor 33 cancels the contract procedure in accordance with the cancellation reply notification.

Furthermore, when requesting execution of processing of the contract procedure in response to the cancellation confirmation notification, the user (or the guardian) may operate the operation part 21 of the terminal 20 to make a reply with an execution request notification in response to the cancellation confirmation notification.

In such a case, the processor 22 receives an instruction to make a reply with the execution request notification from the operation part 21, and creates the execution request notification for requesting execution of processing of the contract procedure. The communicator 23 transmits the created execution request notification to the server 30. The execution request notification may include the contract information that identifies the contract procedure to be executed, and may include identification information that identifies the user (for example, the user ID).

The communicator 31 of the server 30 receives the execution request notification and outputs the received execution request notification to the contract processor 33. When the communicator 31 has not received the cancellation reply notification from the terminal 20 within a prescribed reconfirmation period after transmission of the cancellation confirmation notification to the terminal 20 or the communicator 31 has received the execution request notification from the terminal 20, the contract processor 33 executes processing of the contract procedure. Furthermore, the cancellation confirmation notification is a notification for confirming cancellation of the contract procedure with the user before the grace period has elapsed, and an optional period thus may be set to the reconfirmation period as long as the reconfirmation period is within the grace period.

Furthermore, the server 30 may transmit the cancellation confirmation notification to the terminal 10. In such a case, the contract processor 33 makes an inquiry to the estimator 34 about whether the biological data of the user performing the contract procedure that is recorded in the history information storage 32 deviates from an allowable range. When the piece of biological data of the user performing the contract procedure that is recorded in the history information storage 32 deviates from an allowable range, the contract processor 33 creates the cancellation confirmation notification for confirming, with the user, whether to cancel the contract procedure indicated by the piece of contract information recorded in association with the deviating piece of biological data deviating from the allowable range. The communicator 31 transmits the created cancellation confirmation notification to the terminal 10.

The communicator 15 of the terminal 10 receives the cancellation confirmation notification from the server 30 and outputs the received cancellation confirmation notification to the processor 13. The processor 13 displays the cancellation confirmation notification on the display 14 to present the cancellation confirmation notification to the user.

When the user requests cancellation of the contract procedure recorded in association with the deviating piece of biological data in response to the cancellation confirmation notification, the user operates the operation part 11 to make a reply with the cancellation reply notification in response to the cancellation confirmation notification. The processor 13 receives an instruction to make a reply with the cancellation reply notification from the operation part 11, and creates the cancellation reply notification for requesting cancellation of the contract procedure. The communicator 15 transmits the created cancellation reply notification to the server 30.

The communicator 31 of the server 30 receives the cancellation confirmation notification and outputs the received cancellation confirmation notification to the contract processor 33. When the communicator 31 has received the cancellation reply notification, the contract processor 33 cancels the contract procedure in accordance with the cancellation reply notification.

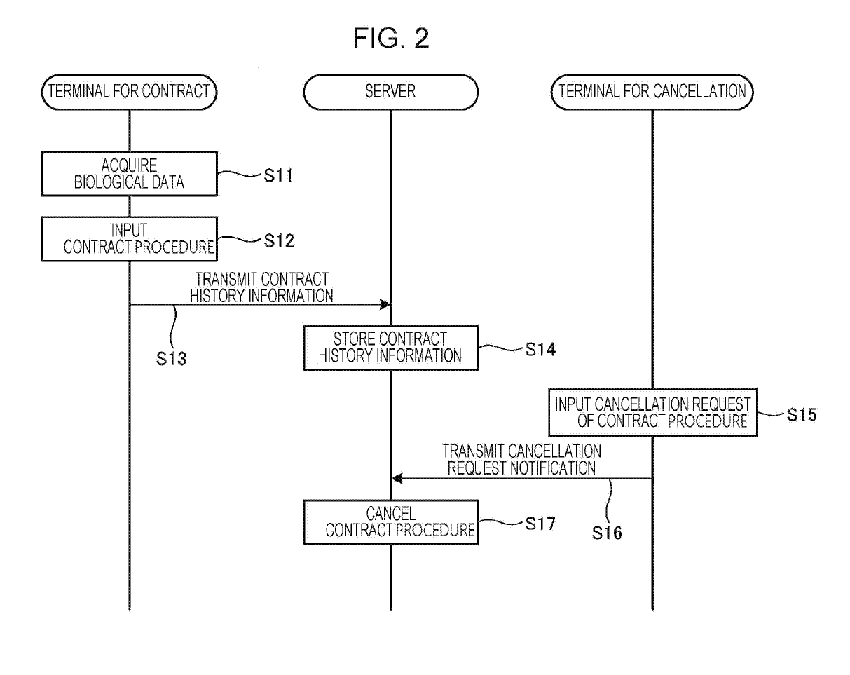

Next, contract cancellation processing executed by the contract cancellation system configured as described above will be described. FIG. 2 is a sequence diagram illustrating an example of contract cancellation processing executed by the contract cancellation system illustrated in FIG. 1. It is to be noted that in the description below, a case where the user performs the contract procedure using the terminal 10 and then uses the terminal 20 to perform cancellation of the contract procedure will be described as an example.

As illustrated in FIG. 2, firstly, the user starts the contract procedure using the terminal 10. The biological data measurer 12 then measures biological data of the user performing the contract procedure by operating the operation part 11 to acquire the biological data and outputs the acquired biological data to the processor 13 (Step S11).

Next, the operation part 11 receives an input operation of the contract procedure by the user and outputs the contract information indicating the contract content of the contract procedure to the processor 13 (Step S12).

Next, the processor 13 generates the contract history information associating the contract information indicating the contract content of the contract procedure that has been input by the user by operating the operation part 11, the time information indicating the time of performing the contract procedure, and the biological data at the time of performing the contract procedure, and outputs the generated contract history information to the communicator 15. The communicator 15 then transmits the contract history information to the server 30 (Step S13).

Next, the communicator 31 of the server 30 receives the contract history information transmitted from the terminal 10, and records the received contract history information in the history information storage 32 (Step S14).

Thereafter, when the user reconsider the contract procedure in a cool state of mind and desires to cancel the contract procedure, the operation part 21 receives an input operation of the cancellation request notification for requesting cancellation of the contract procedure by the user and instructs the processor 22 to create the cancellation request notification (Step S15).

Next, the processor 22 creates the cancellation request notification in accordance with an instruction from the operation part 21, and the communicator 23 transmits the created cancellation request notification to the server 30 (Step S16).

Finally, the communicator 31 of the server 30 receives the cancellation request notification from the terminal 20 and outputs the received cancellation request notification to the contract processor 33. When the communicator 31 has received the cancellation request notification within the grace period, the contract processor 33 identifies the piece of contract information corresponding to the contract procedure whose cancellation is requested by the cancellation request notification from the contract history information recorded in the history information storage 32. When the piece of biological data associated with the identified piece of contract information deviates from a prescribed allowable range, the contract processor 33 cancels the contract procedure in accordance with the cancellation request notification (Step S17).

With the above-described processing, for example, in an electronic settlement procedure, procedures such as remittance or transfer are canceled. In an electronic commerce procedure, procedures such as buying and selling of a product or a service are canceled.

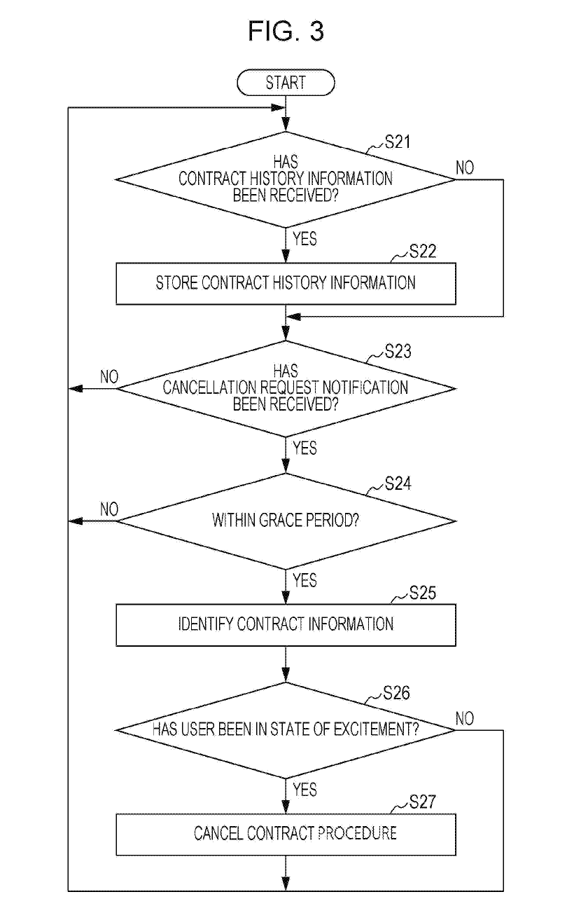

Next, server contract cancellation processing executed by the server 30 out of the above-described contract cancellation processing will be described in more details. FIG. 3 is a flowchart illustrating an example of the server contract cancellation processing executed by the server 30 illustrated in FIG. 1.

Firstly, the communicator 31 of the server 30 determines whether the communicator 31 has received the contract history information transmitted from the terminal 10 (Step S21). When the communicator 31 has not received the contract history information (No at Step S21), the processing proceeds to Step S23.

On the other hand, when the communicator 31 has received the contract history information (Yes at Step S21), the communicator 31 records the received contract history information in the history information storage 32 (Step S22).

Next, the communicator 31 determines whether the communicator 31 has received the cancellation request notification from the terminal 20 (Step S23). When the communicator 31 has not received the cancellation request notification (No at Step S23), the processing proceeds to Step S21 and continues the processing at and after Step S21.

On the other hand, when the communicator 31 has received the cancellation request notification (Yes at Step S23), the communicator 31 outputs the received cancellation request notification to the contract processor 33. The contract processor 33 determines whether the communicator 31 has received the cancellation request notification within the grace period (Step S24).

When the communicator 31 has not received the cancellation request notification within the grace period (No at Step S24), the processing proceeds to Step S21 and continues the processing at and after Step S21. It is to be noted that this disables cancellation of the contract procedure, and the contract processor 33 thus may use the communicator 31 to notify the terminal 20 that cancellation of the contract procedure is disabled.

On the other hand, when the communicator 31 has received the cancellation request notification within the grace period (Yes at Step S24), the contract processor 33 identifies the piece of contract information corresponding to the contract procedure whose cancellation is requested by the cancellation request notification from the contract history information recorded in the history information storage 32 and outputs the identified piece of contract information to the estimator 34 (Step S25).

Next, the estimator 34 reads out the piece of biological data associated with the identified piece of contract information from the history information storage 32 and estimates, from the piece of biological data associated with the identified piece of contract information, whether the state of mind of the user at the time of performing the contract procedure is a state of excitement (Step S26).

When it is estimated that the state of mind of the user at the time of performing the contract procedure is not a state of excitement (No at Step S26), the processing proceeds to Step S21 and continues the processing at and after Step S21. It is to be noted that this also disables cancellation of the contract procedure, and the estimator 34 thus may notify the contract processor 33 of the estimation result, and the contract processor 33 may use the communicator 31 to notify the terminal 20 that cancellation of the contract procedure is disabled.

On the other hand, when it is estimated that the state of mind of the user at the time of performing the contract procedure is a state of excitement (Yes at Step S26), the estimator 34 notifies the contract processor 33 of the estimation result, and the contract processor 33 deletes, from the history information storage 32, the contract history information including the piece of contract information used for making the estimation that the state of mind of the user at the time of performing the contract procedure is a state of excitement, thereby canceling the contract procedure in response to the cancellation request notification (Step S27). Thereafter, the processing proceeds to Step S21 and continues the processing at and after Step S21. It is to be noted that this has enabled cancellation of the contract procedure, and the contract processor 33 thus may use the communicator 31 to notify the terminal 20 that cancellation of the contract procedure has been completed.

With the above-described processing, according to the present embodiment, when the server 30 has received the cancellation request notification within the grace period from the terminal 20, the piece of contract information corresponding to the contract procedure whose cancellation is requested by the cancellation request notification is identified from the contract history information recorded in the history information storage 32. Furthermore, when a state of excitement of the user has been estimated from the piece of biological data associated with the identified piece of contract information, the contract procedure is canceled in accordance with the cancellation request notification. As described above, only when a state of excitement of the user has been estimated from the piece of biological data of the user performing the contract procedure, the contract procedure is canceled after the contract procedure has been performed. With this, only when it is accurately detected that the user has performed the contract procedure by mistake in an inappropriate state of mind, the contract procedure can be canceled after the user has performed the contract procedure. Consequently, when the user has performed the contract procedure by mistake in an inappropriate state of mind, the contract procedure can be appropriately handled after the user has performed the contract procedure.

The contract cancellation system according to an aspect of the present disclosure has been described above based on the embodiment described above. However, the present disclosure is not limited to the embodiment described above. Various modifications that the skilled person could have arrived at may be applied to the embodiment, or a component in a different embodiment may be combined with the embodiment. An embodiment thus formed is included in the scope of the present disclosure unless the embodiment deviates from the object of the present disclosure.

With the contract cancellation system, the server, and the contract cancellation method according to the present disclosure, when a user has performed a prescribed contract procedure by mistake in an inappropriate state of mind, the user can appropriately handle the contract procedure after performing the contract procedure. The contract cancellation system, the server, and the contract cancellation method according to the present disclosure thus is effective for canceling a contract procedure performed by the user, for example, for canceling an electronic settlement procedure or an electronic commerce procedure.

What is claimed is:

1. A contract cancellation system comprising:
a first communication terminal;
a second communication terminal; and
a server, wherein
the first communication terminal includes
a first operation part operated by a first user,
an acquirer that acquires biological data of the first user, and
a first transmitter that transmits, to the server, contract history information in which contract information indicating contract content of a contract procedure, time information indicating a time of performing the contract procedure, and the biological data at the time of performing the contract procedure are associated with one another, when the first user has performed the contract procedure on the first operation part,
the second communication terminal includes
a second operation part operated by a second user, and
a second transmitter that transmits, to the server, a cancellation request notification for requesting cancellation of the contract procedure, when the second user has performed an operation for canceling the contract procedure on the second operation part,
the server includes
a first receiver that receives the contract history information,
a recorder that records the contract history information received by the first receiver, and
a contract processor that executes processing of the contract procedure after a first period has elapsed from a time of recording the contract history information by the recorder, and
the contract processor cancels the contract procedure, when the first receiver has received the cancellation request notification within the first period and the biological data associated with the contract information deviates from an allowable range.

2. The contract cancellation system according to claim 1, wherein
the contract procedure is an electronic settlement procedure, and
the contract processor cancels the electronic settlement procedure, when the first receiver has received the cancellation request notification within the first period and the biological data associated with the contract information deviates from the allowable range.

3. The contract cancellation system according to claim 1, wherein
the contract procedure is an electronic commerce procedure, and
the contract processor cancels the electronic commerce procedure, when the first receiver has received the cancellation request notification within the first period and the biological data associated with the contract information deviates from the allowable range.

4. The contract cancellation system according to claim 1, wherein
the server further includes an estimator that estimates a state of excitement of the first user by analyzing the biological data, and
the contract processor cancels the contract procedure, when the first receiver has received the cancellation request notification within the first period and the estimator estimates that the first user has been in a state of excitement, based on the biological data associated with the contract information.

5. The contract cancellation system according to claim 4, wherein
the estimator estimates that the first user has been in a state of excitement, when a difference between first reference biological data being an average value of biological data of the first user in a normal state and the biological data is equal to or higher than a prescribed value.

6. The contract cancellation system according to claim 4, wherein
the estimator estimates that the first user has been in a state of excitement, when a difference between second reference biological data being an average value of biological data of a plurality of users in a normal state and the biological data is equal to or higher than a prescribed value.

7. The contract cancellation system according to claim 1, wherein
the contract processor creates a cancellation confirmation notification for confirming whether to cancel the contract procedure with the second user after a second period has elapsed from the time of recording the contract history information by the recorder, when the biological data associated with the contract information deviates from the allowable range,
the server further includes a third transmitter that transmits the cancellation confirmation notification to the second communication terminal,
the second communication terminal further includes a second receiver that receives the cancellation confirmation notification,
the second transmitter transmits, to the server, a cancellation reply notification for requesting cancellation of the contract procedure, when the second user has performed an operation for canceling the contract procedure on the second operation part in response to the cancellation confirmation notification, the second transmitter transmits, to the server, and
the contract processor cancels the contract procedure, when the first receiver has received the cancellation reply notification.

8. The contract cancellation system according to claim 7, wherein
the second transmitter transmits, to the server, an execution request notification requesting execution of the contract procedure, when the second user has performed an operation for requesting execution of the contract procedure on the second operation part in response to the cancellation confirmation notification, and
the contract processor executes the processing of the contract procedure, when the contract processor has not received the cancellation reply notification from the second communication terminal within the second period from the time of transmitting the cancellation confirmation notification to the second communication terminal or has received the execution request notification from the second communication terminal.

9. The contract cancellation system according to claim 1, wherein
the contract processor creates a cancellation confirmation notification for confirming whether to cancel the contract procedure with the first user, when the biological data associated with the contract information deviates from the allowable range,
the server further includes a third transmitter that transmits the cancellation confirmation notification to the first communication terminal,
the first communication terminal further includes a third receiver that receives the cancellation confirmation notification,
the first transmitter transmits, to the server, a cancellation reply notification for requesting cancellation of the contract procedure, when the first user has performed an operation for canceling the contract procedure on the first operation part in response to the cancellation confirmation notification, and
the contract processor cancels the contract procedure, when the first receiver has received the cancellation reply notification.

10. The contract cancellation system according to claim 1, wherein the first communication terminal and the second communication terminal are the same communication terminal.

11. The contract cancellation system according to claim 1, wherein the first user and the second user are the same user.

12. The contract cancellation system according to claim 1, wherein the acquirer acquires the biological data from a wearable terminal worn by the first user.

13. The contract cancellation system according to claim 1, wherein the acquirer includes a sensor for measuring the biological data.

14. A server communicably connected to a first communication terminal and a second communication terminal, comprising:
a receiver that receives, from the first communication terminal, contract history information in which contract information indicating contract content of a contract procedure performed by a first user using the first communication terminal, time information indicating a time of performing the contract procedure, and biological data of the first user at the time of performing the contract procedure are associated with one another;
a recorder that records the contract history information received by the receiver; and
a contract processor that executes processing of the contract procedure after a first period has elapsed from a time of recording the contract history information by the recorder, wherein
the contract processor cancels the contract procedure, when the receiver has received the cancellation request notification for requesting cancellation of the contract procedure within the first period from the second communication terminal and the biological data associated with the contract information deviates from an allowable range.

15. A contract cancellation method comprising:
receiving contract history information in which contract information indicating contract content of a contract procedure performed by a first user, time information indicating a time of performing the contract procedure, and biological data of the first user at the time of performing the contract procedure are associated with one another,
recording the received contract history information in a recorder,
canceling the contract procedure, when a cancellation request notification by a second user for requesting cancellation of the contract procedure has been received within a first period from a time of recording the contract history information in the recorder and the biological data associated with the contract information deviates from an allowable range, and
executing the contract procedure after a first period has elapsed, when the cancellation request notification has not been received within the first period.
